(19) United States
(12) Patent Application Publication
(10) Pub. No.: US 2012/0064159 A1
(43) Pub. Date: Mar. 15, 2012

(54) Multilayer Oral Tablets Containing a Non-Steroidal Anti-Inflammatory Drug and/or Acetaminophen

(75) Inventors: Ishwar Chauhan, Voorhees, NJ (US); Siva Rama Krishna Nutalapati, Princeton, NJ (US)

(73) Assignee: Aptapharma, Inc., Pennsauken, NJ (US)

(21) Appl. No.: 13/321,575

(22) PCT Filed: May 24, 2010
(86) PCT No.: PCT/US10/35926

§ 371 Date: Nov. 21, 2011

Related U.S. Application Data
(60) Provisional application No. 61/181,745, filed on May 28, 2009.

(51) Int. Cl. A61K 9/42; A61P 29/00; B29C 65/76; A61K 31/167

(52) U.S. Cl. 424/465; 514/629; 156/306.3

(57) ABSTRACT

Multilayer tablets of a non-steroidal anti-inflammatory drug (NSAID) and/or acetaminophen for oral administration containing an immediate release layer or layers containing a NSAID and/or acetaminophen and/or a second therapeutic agent and an extended release layer containing a NSAID and/or acetaminophen are provided. Multilayer tablets containing an additional immediate and/or extended release layer of a second therapeutic agent are also provided. Methods for production of these multi-layer tablets and methods for their use in treating a subject in need of a NSAID and/or acetaminophen are also provided.

This patent application claims the benefit of priority from U.S. Provisional Application Ser. No. 61/181,745, filed May 28, 2009, teachings of which are herein incorporated by reference in their entirety.

### FIELD OF THE INVENTION

The present invention relates to multilayer oral tablets of a non-steroidal anti-inflammatory drug (NSAID) and/or acetaminophen, containing one or more immediate release layers and one or more extended release layers.

### BACKGROUND OF THE INVENTION

Multilayer controlled-release tablets comprising an immediate release layer and an extended release layer of an active agent such as a non-steroidal anti-inflammatory drug (NSAID) or acetaminophen are described in U.S. Pat. No. 5,681,583, U.S. Pat. No. 6,372,255, and U.S. Pat. No. 5,073,380.

### SUMMARY OF THE INVENTION

An aspect of the present invention relates to a multilayer tablet of a non-steroidal, anti-inflammatory drug (NSAID) and/or acetaminophen comprising one or more immediate release layers containing a NSAID and/or acetaminophen and one or more extended release layers containing a NSAID and/or acetaminophen.

In one embodiment, the multilayer tablet further comprises a second therapeutic agent in an immediate release layer and/or an extended release layer.

Another aspect of the present invention relates to a method for formulating a NSAID and/or acetaminophen as a multilayer tablet comprising one or more immediate release layers containing a NSAID and/or acetaminophen and one or more extended release layers containing a NSAID and/or acetaminophen.

In one embodiment of this method, the method further comprises formulating a second therapeutic agent in an immediate release and/or extended release layer. Another aspect of the present invention relates to a method for treating a subject in need of a NSAID and/or acetaminophen which comprises administering to the subject a multilayer tablet of a NSAID and/or acetaminophen comprising one or more immediate release layers containing a NSAID and/or acetaminophen and one or more extended release layers containing a NSAID and/or acetaminophen.

In one embodiment of this treatment method, the multilayer tablet administered further comprises an additional therapeutic agent in an immediate release layer and/or an extended release layer.

### DETAILED DESCRIPTION OF THE INVENTION

The present invention provides multilayer tablets of a non-steroidal anti-inflammatory drug and/or acetaminophen for oral administration.

Non-steroidal anti-inflammatory drugs (NSAIDs), also referred to in the literature as non-steroidal anti-inflammatory agents/analgesics (NSAIAs) or non-steroidal anti-inflammatory medicines, are drugs with analgesic, antipyretic (lowering an elevated body temperature and relieving pain without impairing consciousness) and, in higher doses, anti-inflammatory effects. The term “non-steroidal” is used to distinguish these drugs from steroids with similar effects. Examples of NSAIDs include, but are in no way limited to, aspirin, ibuprofen, and naproxen. By NSAID as used herein it meant to include any agent with these effects as well as pharmaceutically acceptable salts thereof.

For example, the propionic acid derivative ((S)-6-methoxy-methyl-2-naphthaleneacetic acid), Naproxen, is a NSAID. Naproxen is commonly used as either its free acid or its sodium salt, naproxen sodium. Naproxen exhibits analgesic and antipyretic properties and is used to relieve mild to moderately severe pain in rheumatoid arthritis, osteoarthritis and other inflammatory conditions.

The effects of naproxen are associated with the inhibition of prostaglandin synthesis and in particular cyclooxygenase, an enzyme that catalyzes the formation of prostaglandin precursors from arachidonic acid.

Naproxen and naproxen sodium are generally administered two to four times daily. Plasma naproxen concentrations of 30-90 μg/ml reportedly are required for anti-inflammatory or analgesic effects.

An exemplary, but not limiting, pharmaceutically acceptable salt used in the multilayer tablets of the present invention is the sodium salt of naproxen, also referred to as naproxen sodium. However, as will be understood by the skilled artisan upon reading this disclosure, alternative pharmaceutically acceptable salts of any NSAID can be used and are encompassed by the present invention. When using the term naproxen in the detailed description, it should be understood to include embodiments of tablets comprising the propionic acid derivative ((S)-6-methoxy-methyl-2-naphthaleneacetic acid) and/or pharmaceutically acceptable salts thereof.

Acetaminophen, also referred to as paracetamol is another widely used over-the-counter analgesic and antipyretic useful in the multilayer tablets of the present invention. Acetaminophen is commonly used for the relief of fever, headaches, and other minor aches and pains, and is a major ingredient in numerous cold and flu remedies. In combination with non-steroidal anti-inflammatory drugs (NSAIDs) or opioid analgesics, paracetamol is used also in the management of more severe pain (such as cancer pain). Tablets of the present invention can be prepared by methods well-known in the art. Generally recognized compendiums of such methods include Remington: The Science and Practice of Pharmacy, Alfonso R. Gennaro, editor, 20th ed. Lippincott Williams & Wilkins: Philadelphia, Pa., 2000 and Sheth et al. Compressed Tablets, in Pharmaceutical Dosage Forms: Tablets, Vol 1. edited by H. A. Lieberman and L. Lachman, Dekker N.Y. (1980).

In one embodiment of the present invention, the multilayer tablet of the present invention is formulated to release from one or more immediate release layers a first predetermined amount of NSAID and/or acetaminophen immediately to a subject. upon administration and to release from one or more extended release layers a second predetermined amount of NSAID and/or acetaminophen over an extended time period following administration to the subject. Each multilayer tablet comprises one or more NSAID and/or acetaminophen containing immediate release layers and one or more NSAID and/or acetaminophen containing extended release layers.

In simplest form, this tablet embodiment of the present invention is a bilayer tablet comprising a single NSAID and/or acetaminophen containing immediate release layer and a single NSAID and/or acetaminophen containing extended release layer. As will be understood by the skilled artisan upon reading this disclosure, tablets of the present invention may comprise additional NSAID and/or acetaminophen containing immediate release layers and/or additional NSAID and/or acetaminophen containing extended release layers.

The immediate release layer or layers is that part of the multilayer tablet with a dissolution profile from 0 to 60 minutes in a suitable in vitro dissolution test. A suitable exemplary dissolution test is set forth in Example 11 herein. In this exemplary test, dissolution is carried out in 900 mL of phosphate buffer (pH 6.8) at a temperature of 37.0° C±0.5° C. using USP type II dissolution apparatus (paddles) rotating at a speed of 75 rpm. However, as will be understood by the skilled artisan upon reading this disclosure, variations on this test as well as the apparatus and conditions well known to those skilled in the art can be used. In one embodiment of the present invention, 25-100% of the NSAID and/or acetaminophen in the immediate release layer or layers is dissolved in 60 minutes and more preferably in 30 minutes, in a suitable in vitro dissolution test, such as described herein in Example 11.

The extended release layer or layers of the multilayer tablet of the present invention is that part of the tablet with a dissolution profile which is after 30 minutes, measured in a suitable in vitro dissolution test, such as described herein in Example 11. In one embodiment of the present invention, the complete dissolution time of the NSAID and/or acetaminophen in the extended release layer or layers is within 12 hours, in a suitable in vitro dissolution test, such as described herein in Example 11.

In this embodiment, the tablet may further comprise an additional layer or layers comprising a second therapeutic agent. By “second therapeutic agent” it is meant an additional active pharmaceutical ingredient different from the NSAID and/or acetaminophen. The additional layer or layers comprising the second therapeutic agent may be an immediate release layer or layers or an extended release layer or layers.

In this tablet embodiment of the present invention, the NSAID and/or acetaminophen containing immediate release layer or layers comprises the NSAID and/or acetaminophen and a substituted alkyl cellulose.

Suitable substituted alkyl celluloses for the immediate release layer include, but are not limited to, hydroxy or carboxy substituted alkyl celluloses (e.g., hydroxyl propylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), and combinations thereof comprising at least one of the foregoing.

The immediate release layer may optionally comprise additional excipients such as binders, fillers, disintegrants, lubricants, glidants, and the like.

Suitable fillers to be optionally included include carbohydrate or protein fillers such as, but not limited to, sugars, including lactose, sucrose, mannitol, and sorbitol, starch from, for example, corn, wheat, rice, potato, and other plants, cellulose derivatives such as microcrystalline cellulose, gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

A disintegrant or disintegrants may be optionally included in the immediate release layer to facilitate the breakdown of the immediate release layer in a fluid environment, specifically an aqueous fluid environment. The choice and amount of disintegrant can be tailored to ensure the desired dissolution profile of the formulation in vivo. Exemplary disintegrants include a material that possesses the ability to swell or expand upon exposure to a fluid environment, especially an aqueous fluid environment. Exemplary disintegrants include, but are not limited to, hydroxyl substituted alkyl celluloses (e.g., hydroxypropyl cellulose), starch, pregelatinized starch (e.g., Starch 1500® available from Colorcon); cross-linked sodium carboxymethylcellulose (“croscarmellose sodium”, i.e., Ac-Di-Sol® available from FMC BioPolymer of Philadelphia, Pa.); crosslinked homopolymer of N-vinyl-2-pyrrolidone (“crospovidone”, e.g., Polyplasdone® XL, Polyplasdone® XL-10, and Polyplasdone® INF-10 available from International Specialty Products, Wayne N.J.); modified starches, such as sodium carboxymethyl starch, sodium starch glycolate (e.g., Primogel), and the like; alginates; and combinations comprising at least one of the foregoing.

The amount of disintegrant used depends upon the disintegrant or disintegrant combination chosen and the targeted release profile of the resulting formulation. Exemplary amounts include, about 0 to about 10 wt. % based on the total weight of the immediate release layer, specifically about 0.1 to about 7.0 wt. %, and yet more specifically about 0.5 to about 5.0 wt. %.

Exemplary lubricants which may optionally be included in the immediate release layer include, but are not limited to, stearic acid, stearates (e.g., calcium stearate, magnesium stearate, and zinc stearate), sodium stearyl fumarate, glycerol behenate, mineral oil, polyethylene glycols, talc, hydrogenated vegetable oil, vegetable based fatty acids, and a combination comprising at least one of the foregoing. Glidants which may be optionally included are, for example, a silicon dioxide (e.g. fumed or colloidal). It is recognized by those skilled in the art that certain materials can function both as a glidant and a lubricant.

The lubricant or glidant can be used in amounts of about 0.1 to about 15 wt. % of the total weight of the immediate release layer; specifically about 0.2 to about 5 wt. %; and yet more specifically about 0.5 to about 3 wt. %.

The NSAID and/or acetaminophen containing extended release layer or layers of this tablet embodiment comprises a NSAID and/or acetaminophen and a wax excipient.

By wax excipient it is meant to include wax and wax-like excipients and combinations thereof. Exemplary wax excipients for use in the present invention include, but not limited to, carnauba wax (from the palm tree Copernicia Cerifera), vegetable wax, fruit wax, microcrystalline wax (“petroleum wax”), bees wax (white or bleached, and yellow), hydrocarbon wax, paraffin wax, cetyl esters wax, non-ionic emulsifying wax, anionic emulsifying wax, candelilla wax, and combinations thereof comprising at least one of the foregoing waxes. Other suitable wax-like excipients useful in the present invention include, but are not limited to, fatty alcohols (such as lauryl, myristyl, stearyl, cetyl or specifically cetostearyl alcohol), hydrogenated vegetable oil, hydrogenated castor oil, fatty acids such as stearic acid, fatty acid ethers, fatty acid esters including fatty acid glycerides (mono-, di-, and tri-glycerides), polyethylene glycol (PEG) having a molecular weight of greater than about 3000 number average molecular weight, Mₙ (e.g. PEG 3350, PEG 4000, PEG 4600, PEG 6000, and PEG 8000), cremophore and combinations thereof comprising at least one of the foregoing. Any combination of wax and wax-like excipients are contemplated by the present invention.

The amount of wax excipient present in the extended release layer can be determined based on the particular wax or wax combination chosen and the targeted release profile desired for the resulting formulation. Exemplary amounts of a wax excipient include about 5 to about 80 wt. % based on the total weight of the extended release layer, specifically about 10 to about 75 wt. %, and more specifically about 15 to about 70 wt. %.

The extended release layers used in the tablets of the present invention do not contain acrylic polymers.

The extended release layer may optionally further contain an additional release-retarding material. Examples of additional release-retarding materials include, but are not limited to, an alkylcellulose including substituted alkylcellulose, shellac, zein, polyvinylpyrrolidine including crosslinked polyvinylpyrrolidinone, a polyethylene oxide, and combinations thereof comprising at least one of the foregoing materials.

Suitable alkylcelluloses include, for example, methylcellulose, ethylcellulose, and the like. Those skilled in the art will appreciate that other cellulosic polymers, including other alkyl cellulosic polymers, can be substituted for part or all of the ethylcellulose. Other release-retarding matrix materials include modified celluloses such as a carboxymethylcellulose, a low molecular weight hydroxypropylmethylcellulose, a medium viscosity hydroxypropylmethylcellulose, a crosslinked sodium carboxymethylcellulose, a crosslinked hydroxypropylcellulose, a high molecular weight hydroxypropylmethylcellulose, or a combination comprising at least one of the foregoing materials.

The additional release-retarding material can be present in the extended release layer in an amount of 0 to about 75 wt. % based on the total weight of the extended release layer, specifically about 0.1 to about 70 wt. %, more specifically about 1 to about 65 wt. %.

The extended release layer may optionally further comprise an organic acid, binders, fillers, disintegrants, lubricants, glidants, and the like.

In this embodiment, the core can comprise an organic acid, the NSAID and/or acetaminophen. The organic acid, when such is used, is preferably selected from adipic acid, ascorbic acid, citric acid, fumaric acid, malic acid, succinic acid or tartaric acid. The NSAID and/or acetaminophen component and the organic acid when present, are preferably present in the ratio of from 50:1 to 1:50.

Exemplary optional lubricants include, but are not limited to, stearic acid, stearates (e.g., calcium stearate, magnesium stearate, and zinc stearate), sodium stearyl fumarate, glycerol behenate, mineral oil, polyethylene glycols, talc, hydrogenated vegetable oil, vegetable-based fatty acids, or combinations thereof comprising at least one of the foregoing.

Exemplary optional glidants include, but are not limited to, silicon dioxides (e.g. fumed or colloidal). It is recognized by those skilled in the art that certain materials can function both as a glidant and a lubricant.

The lubricant or glidant can be used in amounts of about 0.1 to about 15 wt. % of the total weight of the extended release layer; specifically about 0.5 to about 5 wt. %; and yet more specifically about 0.6 to about 3 wt. %.

The immediate release layer or layers of this tablet embodiment of the present invention are formulated, for example, by preparing a powder mixture by dry blending or granulating or slugging the NSAID and/or acetaminophen with hydroxyalkyl cellulose, adding other optional excipients, if desired, such as fillers, diluents, glidants and lubricants, and then pressing the resulting mixture into a tablet layer or layers.

The extended release layer or layers of this tablet embodiment of the present are formulated, for example, by preparing a powder mixture by dry blending or granulating or slugging the NSAID and/or acetaminophen with a wax excipient, adding other optional excipients, if desired, such as release-retarding materials, fillers, diluents, glidants and lubricants, and pressing the resulting mixture into a tablet layer.

One or more of the immediate release layers and one or more of the extended release layers are compressed together to form a multilayer tablet of the present invention.

In this tablet embodiment of the present invention comprising one or more NSAID and/or acetaminophen containing immediate release layers, 0.1% to 90%, 5% to 85%, or 10% to 80%, of the NSAID and/or acetaminophen is in the immediate release layer or layers and 99% to 10%, 95% to 15%, or 90% to 20%, of the NSAID and/or acetaminophen is in the extended release layer or layers.

In another multilayer tablet embodiment of the present invention, the tablet comprises one or more NSAID and/or acetaminophen containing extended release layers as described supra, and one or more immediate release or extended release layers containing a second therapeutic agent.

The immediate release layer or layers of this tablet embodiment of the present invention are formulated, for example, by preparing a powder mixture by dry blending or granulating or slugging the second therapeutic agent with or without hydroxyalkyl cellulose and a suitable carrier or excipient, adding a lubricant and disintegrant, and then pressing the resulting mixture into a tablet layer or layers.

Examples of second therapeutic agents which can be used in either multilayer tablet embodiment of the present invention include, but are not limited to, analgesics other than NSAIDs and/or acetaminophen, anti-arthritics such as Methotrexate, Sulfasalazine, proton pump inhibitors, drugs used for the treatment of gout such as colchicine, Allopurinol, drugs used for treatment of migraines such as triptans, and ergotamine derivatives. In one embodiment, the second therapeutic agent is a proton pump inhibitor (PPI) such as, but not limited to, Omeprazole, Esomeprazole, Lansoprazole, Pantoprazole and Rabeprazole sodium and/or salts thereof.

In some embodiments, multilayer tablets of the present invention are coated with a film coating polymer. Examples of polymers used for such film coating include, but are not limited to polymers such as polyvinyl pyrrolidine, polyvinyl alcohol, hydroxymethyl cellulose, hydroxypropylcellulose, and hydroxypropylmethylcellulose.

In some embodiments, multilayer tablets of the present invention are coated with an enteric coating.

In embodiments wherein the multilayer tablets are coated, they may optionally include a subcoating to avoid drug interactions with the coating. In these embodiments, prior to applying the enteric or film coating polymer to the tablet, the tablet is coated with a subcoating and then coated with the enteric or film coating polymer. Examples of subcoatings include, but are not limited to polymers such as polyvinyl pyrrolidine, hydroxymethyl cellulose, hydroxypropylmethylcellulose, and hydroxypropylcellulose.

In one embodiment of the present invention, the multilayer tablet exhibits a release rate, when measured in vitro using a USP type II dissolution apparatus (paddle) according to the U.S. Pharmacopoeia in phosphate buffer at pH 6.8 and at 75 rotations per minute, which corresponds to a dissolution pattern of:

from 20 to 70% of total NSAID and/or acetaminophen being released after 1 hour of measurement in the apparatus;

not less than 50% of total NSAID and/or acetaminophen being released after 3 hours of measurement in the apparatus; and

not less than 70% of the total NSAID and/or acetaminophen being released after a total of 6 hours of measurement in the apparatus.

The following nonlimiting examples are provided to further illustrate the present invention.

### EXAMPLES

### Example

Preparation of Naproxen Sodium, 200 mg Immediate Release Tablet Formulation

| Component | Mg/Tab |
|---|---|
| Base granules | |
| Naproxen Sodium | 220 |
| Plasdone K29/32 | 7 |
| SD3A Alcohol * | 0.1 |
| Compression Mix | |
| Base granules | 227 |
| Microcrystalline cellulose (Avicel PH 101) | 128 |
| Hydroxy propylcellulose (Klucel EXF) | 26 |
| Polyplasdone XL | 16 |
| Magnesium Stearate | 3 |
| Total | 400 |

* Not present in final formulation

Naproxen Sodium was weighed. Plasdone K29/32 was dissolved in SD3A alcohol. Naproxen Sodium was then granulated with the Plasdone K29/32 solution. The granules were then dried in an oven at 45° C. and milled using a suitable mill. The Avicel PH 101, Klucel EXF and Polyplasdone XL were passed through a # 20 mesh screen. The screened materials were then blended with the milled Naproxen Sodium granules. The Magnesium stearate was passed through a # 30 mesh screen.

The screened Magnesium Stearate was then added to the above blend and mixed well. 400 mg of the blend was compressed to make Naproxen Sodium immediate release tablets comprising 200 mg of Naproxen.

### Example 2

Preparation of Allopurinol, 100 mg Immediate Release Tablet Formulation

| Component | Mg/Tab |
|---|---|
| Base granules | |
| Allopurinol | 100 |
| Plasdone K29/32 | 3 |
| Purified water * | 0.005 |
| Compression Mix | |
| Base granules | 103 |
| Microcrystalline cellulose (Avicel PH 101) | 50 |
| Lactose mono hydrate | 40 |
| Croscarmellose Sodium (Ac-Di-Sol) | 6 |
| Magnesium Stearate | 1 |
| Total | 200 |

* Not present in final formulation

Allopurinol was weighed. Plasdone K29/32 was dissolved in purified water. Allopurinol was then granulated with Plasdone K29/32 solution. The granules were dried in an oven at 50° C. and milled using a suitable mill. Avicel PH 101, Lactose monohydrate and Ac-Di-Sol were passed through a # 20 mesh screen. The screened materials were blended with milled Allopurinol granules. Magnesium stearate was passed through a # 30 mesh screen. The screened Magnesium Stearate was added to the above blend and mixed well. 200 mg of the blend was compressed to make Allopurinol, 100 mg immediate release tablet.

### Example 3

Preparation of Acetaminophen, 250 mg Immediate Release Tablet Formulation

| Component | Mg/Tab |
|---|---|
| Base granules | |
| Acetaminophen | 250 |
| PVP K29/32 | 8 |
| Purified water* | 0.0085 |
| Compression Mix | |
| Base granules | 258 |
| Microcrystalline cellulose (Avicel PH 101) | 65 |
| Hydroxy propylcellulose (Klucel EXF) | 10 |
| Lactose mono hydrate | 60 |
| Croscarmellose Sodium (Ac-Di-Sol) | 6 |
| Magnesium Stearate | 1 |
| Total | 400 |

*Not present in final formulation

Acetaminophen was weighed. Plasdone K29/32 was dissolved in purified water. Acetaminophen was granulated with Plasdone K29/32 solution. The granules were dried in an oven at 50° C. and milled using a suitable mill. Avicel PH 101, Lactose monohydrate and Ac-Di-Sol were passed through a # 20 mesh screen. The screened materials were then blended with milled Allopurinol granules. Magnesium stearate was passed through a # 30 mesh screen. The screened Magnesium Stearate was added to the above blend and mixed well. 400 mg of the blend was compressed to make Acetaminophen, 250 mg immediate release tablet.

### Example 4

Preparation of Naproxen Sodium, 375 mg Extended Release Tablet Formulation

| Component | Mg/Tab |
|---|---|
| Base granules | |
| Naproxen Sodium | 412.5 |
| Carnauba wax | 227.5 |
| Ethyl cellulose powder (part I) | 40 |
| Ethyl cellulose powder (part II) | 10 |
| SD3A Alcohol* | 0.2 |
| Compression Mix | |
| Base granules | 690 |
| Hydroxy methyl cellulose (Klucel HXF) | 50 |
| Silicon Dioxide (Syloid 244 FP) | 5 |
| Magnesium Stearate | 5 |
| Total | 750 |

*Not present in final formulation

Naproxen Sodium was mixed with Carnauba wax and Ethyl cellulose (part I). Ethyl cellulose (part II) was dissolved in SD3A alcohol. The powder mix was then granulated with the ethyl cellulose solution. The granules were dried in an oven at 45° C. and milled using suitable mill. Kiucel (HXF) and Syloid were passed through a # 20 mesh screen. The milled granules were blended with the screened Kiucel (HXF) and Syloid in a blender. The Magnesium stearate was passed through a # 30 mesh screen. The screened Magnesium Stearate was added to the blender and mixed well. 750 mg of the blend was compressed to make Naproxen Sodium extended release tablets comprising 375 mg of Naproxen.

### Example 5

Preparation of Naproxen Sodium, 375 mg Extended Release Tablet Formulation

| Component | Mg/Tab |
|---|---|
| Base granules | |
| Naproxen Sodium | 412.5 |
| Carnauba wax | 227.5 |
| Ethyl cellulose powder | 50 |
| Stearic acid | 50 |
| SD3A Alcohol* | 0.22 |
| Compression Mix | |
| Base granules | 740 |
| Silicon Dioxide (Syloid 244 FP) | 5 |
| Magnesium Stearate | 5 |
| Total | 750 |

*Not present in final formulation

Naproxen Sodium, Carnauba wax and Ethyl cellulose powder were mixed. Stearic acid was dissolved in SD3A alcohol by heating the alcohol to 50° C. The powder mix was granulated with Stearic acid solution. The granules were dried in an oven at 35° C. and milled using a suitable mill. Syloid was passed through a # 20 mesh screen. The milled granules and screened Syloid were blended in a blender. Magnesium stearate was passed through a # 30 mesh screen. The screened Magnesium Stearate was added to the blender and mixed well. 750 mg of the blend was compressed to make Naproxen Sodium extended release tablets comprising 375 mg of Naproxen.

### Example 6

Preparation of Acetaminophen, 650 mg Extended Release Tablet Formulation

| Component | Mg/Tab |
|---|---|
| Base granules | |
| Acetaminophen | 650 |
| Carnauba wax | 200 |
| Ethyl cellulose powder | 40 |
| Stearic acid | 50 |
| SD3A Alcohol * | 0.25 |
| Compression Mix | |
| Base granules | 940 |
| Silicon Dioxide (Syloid 244 FP) | 5 |
| Magnesium Stearate | 5 |
| Total | 950 |

* Not present in final formulation

Acetaminophen, Carnauba wax and Ethyl cellulose powder were mixed. Stearic acid was dissolved in SD3A alcohol by heating the alcohol to 50° C. The powder mix was granulated with Stearic acid solution. The granules were dried in an oven at 35° C. and milled using a suitable mill. Syloid was passed through a # 20 mesh screen. The milled granules and screened Syloid were blended in a blender. Magnesium stearate was passed through a # 30 mesh screen. The screened Magnesium stearate was added to the blender and mixed well. 950 mg of the blend was compressed to make Acetaminophen, 650 mg extended release tablet.

### Example 7

Preparation of Naproxen Sodium, 500 mg Bi-layer Immediate/Extended Release Tablets

An immediate release formulation was prepared by wet granulation according to Example 1, and an extended release formulation was prepared by wet granulation according to Example 5. The mixtures were then compressed into bi-layer tablets using an alternative tablet press. Each tablet contained 500 mg of Naproxen, the first immediate release layer with 300 mg of blend according to Example 1 comprising 150 mg of Naproxen, and the extended release layer with 700 mg of blend according to Example 5 comprising 350 mg of Naproxen.

### Example 8

### Bi-layer Immediate/Extended Release Tablets Comprising 650 mg Acetaminophen

An immediate release formulation was prepared by wet granulation according to Example 3, and an extended release formulation was prepared by wet granulation according to Example 6. The mixtures were then compressed into bi-layer tablets using an alternative tablet press. Each tablet contained 650 mg Acetaminophen, the first immediate release layer with 400 mg of blend according to Example 3 comprising 250 mg of Acetaminophen, and the extended release layer with 585 mg of blend according to Example 6 comprising 400 mg Acetaminophen.

### Example 9

### Bi-layer Tablets Containing Immediate Release Allopurinol and Extended Release Naproxen Sodium

An immediate release Allopurinol formulation was prepared by wet granulation according to Example 2, and an extended release Naproxen Sodium formulation was prepared by wet granulation according to Example 5. The mixtures were then compressed into bi-layer tablets using an alternative tablet press. Each tablet contained a first immediate release layer with 200 mg of blend according to Example 2 comprising 100 mg of Allopurinol and an extended release layer with 750 mg of blend according to Example 5 comprising 375 mg of Naproxen.

### Example 10

### Tri-layer Immediate/Extended/Immediate Release Tablets Comprising 100 mg of Allopurinol and 500 mg of Naproxen

A first immediate release formulation was prepared by wet granulation according to Example 1. A second immediate release formulation was prepared by wet granulation according to Example 2. An extended release formulation was prepared by wet granulation according to Example 5. The mixtures were then compressed into tri-layer tablets using an alternative tablet press. Each tablet contained 100 mg of immediate release Allopurinol, 150 mg of immediate release Naproxen and 350 mg of extended release Naproxen totaling 500 mg of Naproxen per tablet. The first immediate release layer with 300 mg of blend according to Example 1 comprising 150 mg of Naproxen, the extended release layer with 700 mg of blend according to Example 5 comprising 350 mg Naproxen, and the second immediate release layer with 200 mg of blend according to Example 2 comprising 100 mg of Allopurinol were compressed in the above mentioned sequence to make immediate/extended/immediate release tri-layer tablets comprising 100 mg of Allopurinol and 500 mg of Naproxen.

### Example 11

### Dissolution Profiles of Various Tablets

Dissolution profiles for various tablets, produced in accordance with Examples 1 through 8 were assessed using USP type II dissolution apparatus (paddle) according to the U.S. Pharmacopoeia in phosphate buffer at pH 6.8 and at 75 rotations per minute in 900 ml phosphate buffer. Results are shown in the Table 1 and Table 2.

TABLE 1

| Time | | | |
|---|---|---|---|
| (min) | Ex 1 | Ex 2 | Ex 3 |
| 0 | 0 | 0 | 0 |
| 15 | 70 | 65 | 62 |
| 30 | 95 | 98 | 93 |
| 45 | 96 | 100 | 100 |
| 60 | 98 | 100 | 100 |

TABLE 2

| Time | | | | | |
|---|---|---|---|---|---|
| (hr) | Ex 4 | Ex 5 | Ex 6 | Ex 7 | Ex 8 |
| 0 | 0 | 0 | 0 | 0 | 0 |
| 0.5 | 22 | 20 | 17 | 44 | 50 |
| 0.75 | 27 | 26 | 23 | 48 | 54 |
| 1 | 33 | 30 | 28 | 51 | 57 |
| 1.5 | 43 | 37 | 40 | 56 | 64 |
| 2 | 52 | 43 | 54 | 60 | 72 |
| 4 | 83 | 58 | 85 | 71 | 91 |
| 6 | 95 | 69 | 92 | 78 | 95 |
| 8 | 96 | 79 | 93 | 85 | 96 |
| 10 | 97 | 90 | 94 | 93 | 96 |

1. A multilayer tablet for oral administration comprising:
(a) an immediate release layer or layers comprising a non-steroidal anti-inflammatory drug (NSAID) and/or acetaminophen and a substituted alkylcellulose; and
(b) an extended release layer or layers comprising a NSAID and/or acetaminophen and a wax excipient.

2. The multilayer tablet of claim 1 wherein the extended release layer or layers does not comprise an acrylic polymer.

3. The multilayer tablet of claim 1 wherein the extended release layer or layers further comprises an alkylcellulose or a substituted alkylcellulose.

4. The multilayer tablet of claim 1 wherein the extended release layer or layers does not further comprise an alkylcellulose or a substituted alkylcellulose.

5. The multilayer tablet of claim 1 wherein the extended release layer or layers further comprises an organic acid present in a ratio of NSAID and/or acetaminophen to organic acid of from 50:1 to 1:50.

6. The multilayer tablet of claim 1 further comprising an immediate release layer and/or an extended release layer comprising a second therapeutic agent.

7. The multilayer tablet of claim 6 wherein the second therapeutic agent is a proton pump inhibitor.

8. The multilayer tablet of claim 1 further comprising an enteric coating or a polymer film coating.

9. The multilayer tablet of claim 8 further comprising a subcoating between the tablet and the enteric or polymer film coating.

10. A multilayer tablet for oral administration comprising:
(a) an extended release layer or layers comprising a NSAID and/or acetaminophen and a wax excipient; and
(b) an immediate release layer comprising a second therapeutic agent.

11. The multilayer tablet of claim 10 wherein the extended release layer or layers does not comprise an acrylic polymer.

12. The multilayer tablet of claim 10 wherein the extended release layer or layers further comprises an alkylcellulose or a substituted alkylcellulose.

13. The multilayer tablet of claim 10 wherein the extended release layer or layers does not further comprise an alkylcellulose or a substituted alkylcellulose.

14. The multilayer tablet of claim 10 wherein the second therapeutic agent is a proton pump inhibitor.

15. The multilayer tablet of claim 10 further comprising an enteric coating or a polymer film coating.

16. The multilayer tablet of claim 15 further comprising a subcoating between the tablet and the enteric or polymer film coating.

17. A multilayer tablet of claim 1, wherein the NSAID and/or acetaminophen release rate from the tablet, when measured in vitro using a USP type II dissolution apparatus (paddle) in phosphate buffer at pH 6.8 and at 75 rotations per minute, corresponds to a dissolution pattern of:
a) from 20 to 70% of the total NSAID and/or acetaminophen is released after 1 hour of measurement in said apparatus;
b) not less than 50% of the total NSAID and/or acetaminophen is released after 3 hours of measurement in said apparatus; and
c) not less than 70% of the total NSAID and/or acetaminophen is released after a total of 6 hours of measurement in said apparatus.

18. A method for formulating the multilayer tablet of claim 1 comprising:
(a) preparing an immediate release tablet layer or layers comprising a NSAID and/or acetaminophen and a substituted alkylcellulose;
(b) preparing an extended release tablet layer or layers comprising a NSAID and/or acetaminophen and a wax excipient; and
(c) compressing together the immediate release tablet layer or layers and the extended release tablet layer or layers to form a multilayer tablet.

19. The method of claim 18 further comprising preparing an immediate release and/or extended release layer or layers comprising a second therapeutic agent and compressing together this layer or layers with the immediate release tablet layer or layers and the extended release tablet layer or layers of claim 18.

20. A method for formulating the multilayer tablet of claim 10 comprising:
(a) preparing an extended release tablet layer or layers comprising a NSAID and/or acetaminophen and a wax excipient;
(b) preparing an immediate release tablet layer or layers comprising a second therapeutic agent; and
(c) compressing together the immediate release tablet layer or layers and the extended release tablet layer or layers to form a multilayer tablet.

21. A method for treating a subject in need of a non-steroidal anti-inflammatory agent or acetaminophen comprising administering to the subject the multilayer tablet of claim 1.

22. A multilayer tablet of claim 10, wherein the NSAID and/or acetaminophen release rate from the tablet, when measured in vitro using a USP type II dissolution apparatus (paddle) in phosphate buffer at pH 6.8 and at 75 rotations per minute, corresponds to a dissolution pattern of:
a) from 20 to 70% of the total NSAID and/or acetaminophen is released after 1 hour of measurement in said apparatus;
b) not less than 50% of the total NSAID and/or acetaminophen is released after 3 hours of measurement in said apparatus; and
c) not less than 70% of the total NSAID and/or acetaminophen is released after a total of 6 hours of measurement in said apparatus.

23. A method for treating a subject in need of a non-steroidal anti-inflammatory agent or acetaminophen comprising administering to the subject the multilayer tablet of claim 10.
